(19) United States
(12) Patent Application Publication
(10) Pub. No.: US 2004/0161794 A1
(43) Pub. Date: Aug. 19, 2004

(54) Biosynthesis of HIF

(75) Inventors: Garner T. Haupert JR., Littleton, MA (US); Steven R. Gullans, Natick, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: The General Hospital Corporation, Boston, MA; Brigham and Women's Hospital Inc., Boston, MA

(21) Appl. No.: 10/601,184

(22) Filed: Jun. 20, 2003

Related U.S. Application Data
(60) Provisional application No. 60/390,783, filed on Jun. 20, 2002.

(51) Int. Cl. G01N 33/53

(52) U.S. Cl. 435/7.1

(57) ABSTRACT

The present invention relates to a method of identifying an agent (e.g., peptide, small molecule) that alters (partially, completely) the activity (function, expression) of HIF. In one embodiment, the method of identifying an agent that alters (e.g., inhibits, enhances) the activity of HIF comprises contacting a molecule in the HIF biosynthetic pathway (e.g., a precursor of HIF, such as a steroid precursor; an enzyme) with an agent to be assessed and determining whether the activity of the molecule (function, expression) is altered in the presence of the agent when compared to the activity of the molecule in the absence of the agent. The invention is also related methods of treatment using such agents; and methods of monitoring the biosynthetic pathway of OLC/HIF. In particular, the invention is related to agents, treatments, and diagnostics for diseases such as hypertension and heart failure.

### RELATED APPLICATION(S)

This application claims the benefit of U.S. Provisional Application No. 60/390,783, filed on Jun. 20, 2002, the entire teachings of which are incorporated herein by reference.

### GOVERNMENT SUPPORT

The invention was supported, in whole or in part, by Grant R01 HL52282 from National Institutes of Health/National Heart Lung, and Blood Institute. The Government has certain rights in the invention.

### BACKGROUND OF THE INVENTION

The Na+,K+-ATPase pump (the “Na+ pump”) possesses an evolutionarily conserved binding site. The only known specific regulators of this enzyme in mammals, including humans, are the plant kingdom cardiac glycosides, such as ouabain (Oua), which operate at this conserved binding site. Studies have found Na+ pump inhibitory activity in extracts from body fluid and tissue sources. These inhibitors have been termed “ouabain-like compounds” (OLC), and have been linked to the pathogenesis of hypertension, including experimental volume-expanded hypertension and human essential hypertension (Haber et al., Hypertension, 9:315 (1987); Goto et al., Pharmacol. Rev.,44:377 (1992)). One such OLC, isolated from mammalian hypothalamus, has been termed hypothalamic inhibitory factor (HIF) (Tymiak et al., PNAS, 90:8189(1993); Kawamura, et al., PNAS, 96:6654 (1999); Haupert et al., PNAS, 76:4568 (1979); Haupert, et al., Am. J. Physiol., 247:F919 (1984); Carilli et al., J. Biol. Chem.,260:1027 (1985).

Microscale physiochemical analysis of HIF indicated it was an isomer of Oua, differing in either the point of attachment of the rhamnose sugar moiety to the steroid backbone, or in the stereochemistry of the steroid portion itself (Tymiak et al., PNAS, 90:8189(1993); Kawamura, et al., PNAS, 96:6654 (1999)).

Physiologically, HIF/OLC shares some of the biological properties of plant Oua, but also exhibits important differences. Binding and dissociation of HIF in intact renal tubular cells showed positive cooperativity in binding reactions and a relatively rapid dissociation rate constant, both characteristics different from those of Oua yet consistent with physiologic regulation of the Na+ pump in vivo (Cantiello, et al., Am. J. Physiol.,255:F574 (1988)). HIF was further shown to have positive inotropic effect equipotent with Oua in cultured rat myocytes, at doses nearly 3 orders of magnitude less than those required for Oua (Haupert et al., PNAS, 76:4568 (1979); Haupert, et al., Am. J. Physiol., 247:F919 (1984)). These studies also showed that at the same intracellular Ca++ concentration in myocytes, Oua was toxic whereas HIF was not, indicating less toxicity for HIF versus Oua due to differences in intracellular compartmentalization of Ca++ pools produced by the two inhibitors. Isolated vascular rings from rat pulmonary artery and aorta, unaffected by Oua except at toxic concentrations (rodents are Oua insensitive species), were reversibly and potently constricted by HIF in vitro (Janssens, et al., J. Cardiovasc. Pharmacol., 22:S42 (1993)), consistent with a role of an endogenous Na+,K+-ATPase inhibitor in the regulation of vasoconstriction and pathogenesis of hypertension (Haddy et al., Life Sci., 19:935 (1976); Blaustein et al., Am. J. Physiol., 232 (Cell Physiol. 1):C164 (1977)).

Recent work indicates that mammalian (rat) adrenal tissue can synthesize digitalis-like bioactivity using mammalian steroid pathway precursors (Lichtstein, D., et al., Life Sci., 62:2109-2126 (1998); Perrin, A., et al., Mol. Cell Endocrinol., 126:7-15 (1997); Komiyama, Y., et al., J. Hypertens., 19:229-236 (2001)), and both physiologic and pharmacologic stimuli influence the release or synthesis of HIF/OLC from midbrain and adrenal tissues (De Angelis, C., et al., Am. J. Physiol., 274:F182-F188 (1998); Crabos, M., et al., Am. J. physiol., 254:F912-F917 (1988); Laredo, J., et al., Hypertension, 29:401-407 (1997)). Additionally, neurosteroids can be synthesized in brain tissue either de novo from cholesterol, or from other steroid precursors, and their presence in the nervous system is independent of adrenal gland production. The OLC biosynthetic pathways involved are unknown.

Therefore, there is a need in the art to understand OLC biosynthetic pathways in mammals. Elucidation of such pathways would allow for identification of agents which can modulate the pathways, and thereby modulate inhibition of Na+,K+-ATPase pump, whereby cardiovascular disorders such as hypertension and heart failure can be treated.

### SUMMARY OF THE INVENTION

The present invention relates to a method of identifying an agent (e.g., peptide, small molecule) that alters (partially, completely) the activity (function, expression) of HIF. The method of identifying an agent that alters (e.g., inhibits, enhances) the activity of HIF comprises contacting a molecule in the HIF biosynthetic pathway (e.g., a precursor of HIF, such as a steroid precursor; an enzyme)) with an agent to be assessed, and determining whether the activity of the molecule is altered in the presence of the agent. If the agent alters (partially, completely) the activity of the molecule (function, expression) in the presence of the agent when compared to the activity of the molecule in the absence of the agent, then the agent alters the activity of HIF.

An agent that is effective for modulating the activity of the HIF pathway is an agent that alters the activity of a molecule in the pathway other than HIF (e.g., an enzyme, substrate, intermediate, cofactor, signalling molecule, consumable reagent, and the like). For the activity of the HIF pathway to be modulated, there must be a correlation between the activity of the molecule and the activity of the pathway. In a complex pathway, this correlation can be positive or negative. For example, enhancing the activity of a particular molecule, e.g., a substrate, can enhance the activity of the pathway. Enhancing the activity of another particular molecule, e.g., a molecule that regulates the pathway, can inhibit the HIF pathway.

In one embodiment, the invention relates to a method of identifying an agent that inhibits the activity of HIF, comprising contacting a molecule in the HIF biosynthetic pathway with an agent to be assessed, and determining whether the activity of the molecule is inhibited in the presence of the agent. In one embodiment, if the agent inhibits the activity of the molecule in the presence of the agent when compared to the activity of the molecule in the absence of the agent, then the agent inhibits the activity of HIF. In another embodiment, an agent that enhances the activity of the molecule in the presence of the agent when compared to the activity of the molecule in the absence of the agent, causes inhibition of the activity of HIF.

In another embodiment, the invention relates to a method of identifying an agent that enhances the activity of HIF comprising contacting a molecule in the HIF biosynthetic pathway with an agent to be assessed, and determining whether the activity of the molecule is enhanced in the presence of the agent. In one embodiment, if the agent enhances the activity of the molecule in the presence of the agent when compared to the activity of the molecule in the absence of the agent, then the agent enhances the activity of HIF. In another embodiment, an agent that inhibits the activity of the molecule in the presence of the agent when compared to the activity of the molecule in the absence of the agent, causes enhancement of the activity of HIF.

The present invention also relates to a method of identifying an agent for treating hypertension comprising contacting a molecule in the HIF biosynthetic pathway with an agent to be assessed, and determining whether the activity of the molecule is inhibited in the presence of the agent. If the agent inhibits the activity of the molecule in the presence of the agent when compared to the activity of the molecule in the absence of the agent, then the agent can be used for the treatment of hypertension.

Also encompassed by the present invention is a method of identifying an agent for the treatment of heart failure, comprising contacting a molecule in the HIF biosynthetic pathway with an agent to be assessed, and determining whether the activity of the molecule is enhanced in the presence of the agent. If the agent enhances the activity of the molecule in the presence of the agent when compared to the activity of the molecule in the absence of the agent, then the agent can be used for the treatment of heart failure.

The present invention also relates to the agents identified in the methods described herein.

A molecule in the HIF biosynthetic pathway includes, for example, a precursor of HIF (e.g., a steroid precursor) or an enzyme in the HIF biosynthetic pathway (e.g., P450 side chain cleavage enzyme, Δ5-3β-hydroxysteroid dehydrogenase isomerase).

The agent to be assessed can be, for example, a polypeptide (e.g., L peptide, D peptide) or a small organic molecule. The agent which alters the activity of HIF can alter the function and/or expression of a molecule in the HIF biosynthetic pathway.

There are numerous methods for determining whether the activity of the molecule is altered (inhibited, enhanced) in the presence of the agent which are known to those of skill in the art.

The present invention also provides methods of treating (ameliorating and/or preventing) conditions associated with HIF activity in an individual using the agents identified herein. For example, the present invention provides for a method of treating hypertension comprising administering to an individual in need thereof a therapeutically effective amount of an agent that inhibits the activity of a molecule in a biosynthetic pathway of HIF, using, for example, an agent identified using the methods described herein. In addition, the present invention provides for a method of treating heart failure comprising administering to an individual in need thereof a therapeutically effective amount of an agent that enhances activity of a molecule in a biosynthetic pathway of HIF, using, for example an agent identified using the methods described herein.

Also encompassed by the present invention are diagnostic methods. In one embodiment, the present invention relates to a method of monitoring the effectiveness of a treatment of hypertension in an individual, comprising determining the activity of a molecule in a biosynthetic pathway for hypothalamic inhibitory factor (HIF) in an individual that has been treated, wherein if the activity of the molecule is altered and results in inhibition of HIF activity when the treatment is administered to the individual, compared to the activity of the molecule when the treatment is not administered to the individual, then the treatment is effective.

In another embodiment, the present invention relates to a method for assessing whether an individual is at risk for developing hypertension, comprising determining the activity of a molecule in a subject's biosynthetic pathway for hypothalamic inhibitory factor (HIF), wherein if the activity of the molecule is altered, thereby enhancing the activity of HIF in the individual, then the individual is at risk for developing hypertension.

### BRIEF DESCRIPTION OF THE DRAWINGS

FIG. 1 shows the biosynthetic pathway for conversion of cholesterol to endogenous cardiac glycosides such as HIF. Relevant human genes coding for pathway enzymes are identified by Locus Link numbers and gene symbol. Rat orthologue genes in bold were found upregulated by rat microarray analysis. Rat orthologues italicized were not available on the CodeLink microarray chips. Structures show plant ouabain and candidate precursor molecule.

FIG. 2 shows expression of candidate genes by CodeLink microarray analysis of RNA for hypertensive and normotensive hypothalamus and adrenal tissues. Genes coding for enzymes P450 side chain cleavage (NM_{—}017286; convesion of cholesterol to pregnenolone) and beta hydroxysteroid dehydrogenase isomerase (NM_{—}017265; conversion of pregnenolone to progesterone) are overexpressed in hypertensive hypothalamus but not adrenal. Data are averages of two pairs of rats.

FIG. 3 is a bar graph of the RT-PCR analysis of adult rat hypothalamic and adrenal mRNA. Quantitative (real time) polymerase chain reaction (RT-PCR) analysis of adult Milan hypertensive rat mRNA levels for selected genes (enzymes) in steroid biosynthetic pathway. Data are mean values of four separate analyses for each gene for two sets of paired animals. Data are expressed as fold increase in expression levels in hypertensive vs normotensive (control) animals. SCC, P450 side chain cleavage; βHSD, beta hydroxysteroid dehydrogenase isomerase.

FIG. 4 is a graph showing knock down of HIF activity purified from cultured PC12 (adrenal medulla) cells transfected with siRNA (HSD110) targeting gene Δ-5-3 β-hydroxysteroid dehydrogenase isomerase (NM_{—}017265). HIF was purified from PC12 cell supernatants by column chromatography. Bioactivity is demonstrated by inhibition of Na⁺ pump activity (active Rb⁺ uptake) in human erythrocytes (Carilli et al, 1985). Cells treated with siRNA HSD110 synthesized less HIF as indicated by decrease in inhibitory activity. X-axis reflects optimization studies varying the amount of siRNA (μg) and ratio of siRNA to transfection reagent. Complete inhibition of active Na⁺ transport is caused by 1 mM ouabain as a standard.

FIG. 5 is a graph showing the decrease in Δ-5-3β-hydroxysteroid dehydrogenase isomerase mRNA levels in PC12 (adrenal medulla) cells transfected with siRNA HSD110 as measured by quantitative reverse transcriptase polymerase chain reaction. X-axis reflects optimization studies varying the amount of siRNA (μg) and ratio of siRNA to transfection reagent. Decrease in mRNA was accompanied by decreased production of HIF.

FIG. 6 is an agarose gel showing the clones from subtracted hypertensive hypothalamus.

### DETAILED DESCRIPTION OF THE INVENTION

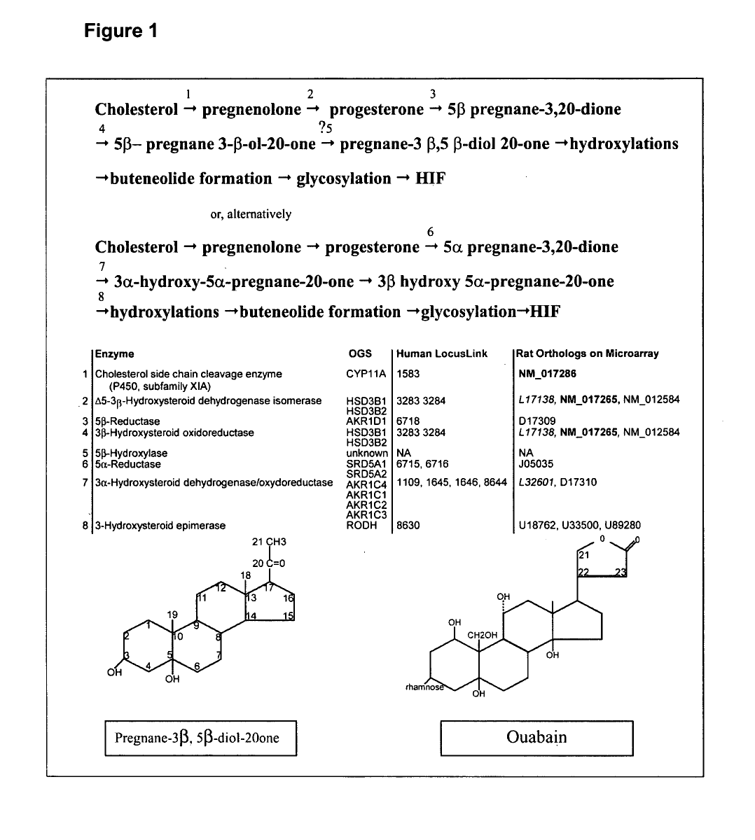

Described herein is generation of the hypothamic inhibitory factor (HIF) biosynthetic pathway(s) (also referred to herein as ouabain like compound (OLC) biosynthetic pathway(s)) and identification of candidate genes encoding enzymes in the pathway(s). Using a bioinformatics approach, steroid biosynthetic pathway(s) leading from cholesterol as a precursor through intermediate hydroxylation steps consistent with generation of a steroid intermediate(s), which after lactone ring addition and glycosylation results in generation of Oua, its isomer, or a related derivativeare described (FIG. 1). Accordingly, the invention is generally related to methods of screening for agents which alter or modulate the biosynthetic pathway of OLC/HIF; methods of treatment using such agents; and methods of monitoring the biosynthetic pathway of OLC/HIF. In particular, the invention is related to agents, treatments, and diagnostics for diseases such as hypertension and heart failure.

In one embodiment, the present invention relates to a method of identifying an agent (one or more) that alters (e.g., modulates) the activity of HIF. The method includes contacting a molecule (one or more) in a biosynthetic pathway for HIF with an agent to be assessed and determining the activity of the molecule in the presence of the agent. If the agent alters the activity of the molecule in the presence of the agent compared to the activity of the molecule in the absence of the agent, then the agent alters the activity of HIF.

As used herein, to “alter” or “modulate”, for example, the activity of a molecule in the HIF biosynthetic pathway or the activity of HIF, includes inhibiting or enhancing the activity of the molecule in the pathway of HIF or the activity of HIF. In one embodiment, the activity of a molecule in the biosynthetic pathway of HIF results in alteration of the activity of HIF.

Thus, in one embodiment, the present invention relates to a method of identifying an agent that inhibits the activity of HIF comprising contacting a molecule in the HIF biosynthetic pathway with an agent to be assessed, and determining whether the activity of the molecule is inhibited in the presence of the agent. In one embodiment, if the agent inhibits the activity of the molecule in the presence of the agent when compared to the activity of the molecule in the absence of the agent, then the agent inhibits the activity of HIF. In another embodiment, an agent that enhances the activity of the molecule in the presence of the agent when compared to the activity of the molecule in the absence of the agent, causes inhibition of the activity of HIF.

As used herein, the term “inhibit” or “inhibiting” encompasses reducing, degrading, downregulating, suppressing, decelerating, or decreasing the activity (e.g., the function or expression) or reaction rate of a molecule in the HIF biosynthetic pathway or HIF. Thus, to “inhibit” the activity of a molecule includes partial or complete inhibition of the activity of the molecule.

In another embodiment, the invention relates to a method of identifying an agent that enhances the activity of HIF comprising contacting a molecule in the HIF biosynthetic pathway with an agent to be assessed, and determining whether the activity of the molecule is enhanced in the presence of the agent. In one embodiment, if the agent enhances the activity of the molecule in the presence of the agent when compared to the activity of the molecule in the absence of the agent, then the agent enhances the activity of HIF. In another embodiment, an agent that inhibits the activity of the molecule in the presence of the agent when compared to the activity of the molecule in the absence of the agent, causes enhancement of the activity of HIF.

The term “enhance” or “enhancing” encompasses improving, increasing, upregulating, promoting, stimulating, upgrading, or accelerating the activity of a molecule in the HIF pathway or HIF. Thus, to “enhance” the activity of a molecule includes partial or complete (e.g., overexpression, reaction rate acceleration) enhancement of the activity of the molecule.

The “activity” of a molecule can be altered in the methods of the present invention by altering, for example, directly or indirectly, the expression or function of the molecule. Altering the expression of a molecule includes increasing or decreasing directly the concentration, synthesis, degradation, digestion, half-life, uptake, excretion, and the like of the molecule. In addition, altering the expression of a molecule includes increasing or decreasing indirectly the concentration, synthesis, degradation, digestion, half-life, uptake, excretion, and the like of a second molecule in the pathway that ultimately has an effect on the molecule. Altering the function of the molecule includes altering the molecule or other molecules it typically interacts with so that the normal effect of the molecule is altered. Such altered function includes, for example, chemical modification such as cleavage, substitution, denaturation, and the like; or kinetic modification, such as competitive binding, reaction rate acceleration/decleration or the like.

As used herein, the term “agent” or “agent to be assessed” includes any compound that can be tested for the claimed effect. Agents typically include small molecules, inorganic and organic molecules, complexes, polymers, and mixtures. More typically, agents include organic molecules and polymers. A particular class of agent includes small organic molecules, e.g., nucleic acids (e.g., antisense RNA, interfering RNA (such as siRNA, shRNA)), peptides, proteins, antibodies (e.g., polyclonal antibody, monoclonal antibody, chimeric antibody, humanized antibody) or fragments thereof (e.g., single chain antibodies, Fv, Fab, Fab′2 fragments), carbohydrates, lipids, steroids, glycosides (e.g., unnatural glycosides), polysachharides, and the like. Other classes of agents include small organic molecules with one or more carbocyclic rings, especially fused polycyclic combinations of cycloalkyl, heterocycloalkyl, aryl, heteroaryl, cycloaliphatic, heterocycloaliphatic and saccharide rings, in particular polycyclic fused ring systems containing a steroid group or fragment thereof. Such agents are typically substituted with functional groups including carboxylic acids, esters, ketones, halogen, nitro, cyano, sulfate, sulfonate, phosphate, phosphonate, amine, amide, carbamate, hydroxyl, C1-C6 alkyl, C1-C6 alkoxy, C1-C6 alkanone, C1-C6 alkanol, and the like. Other functional groups include amino acid groups, sachharides, e.g., hexoses, pentoses, furanoses, and the like. Particular agents include compounds that are stable under physiologic digestive conditions and can be absorbed through digestive tract into the blood stream, and more typically, can pass the blood brain barrier to reach the hypothalamus. Particular agents include competitive substrates, inhibitors, agonists, and antagonists of enzymes in the HIF biosynthetic pathway. One particular class of agents includes L-peptides. Another particular class of agents includes D-peptides.

A molecule in the HIF pathway includes substrates in the pathway, e.g. starting materials, and consumable co-reactants; enzymes that catalyze reactions in the pathway and their associated cofactors; signaling molecules that can alter the pathway or are affected by it, e.g. peptide hormones, metal ions such as calcium, magnesium, sodium, potassium and lithium, nitric oxide, and the like; genes encoding enzymes or peptides in the pathway as polyribonucleic acid fragments or deoxyribonucleic acids; receptors, enzymes, and the like that compete with the HIF pathway for the same molecules (e.g, substrate cofactors); and the like. In a particular embodiment, a molecule in the HIF pathway does not include the product of the pathway, HIF (e.g., the molecule is a molecule other than HIF).

Substrates and intermediates in the HIF pathway include cholesterol, pregnenolone, progesterone, 5-β-pregnane-3,20-dione, 5-β-pregnane-3-β-ol-20-one, pregnane-3-β,5-β-diol 20-one, 5-α-pregnane-3,20-dione, 3-α-hydroxy-5-α-pregnane-20-one, 3-β-hydroxyl-5-α-pregnane-20-one, and fragments, complexes, salts, and chemically substituted derivatives thereof. Other substrates and intermediates in the HIF pathway include cholesterol, pregnenolone, progesterone, 5-β-pregnane-3,20-dione, 5-β-pregnane-3-β-ol-20-one, pregnane-3-β,5-β-diol 20-one, 5-α-pregnane-3,20-dione, 3-α-hydroxy-5-α-pregnane-20-one, and 3-β-hydroxyl-5-α-pregnane-20-one. Particular substrates and intermediates include cholesterol, pregnenolone, progesterone, and 5-β-pregnane-3,20-dione. Other particular substrates and intermediates include pregnenolone, progesterone, and 5-β-pregnane-3,20-dione.

Examples of enzymes in the pathway include a cholesterol side chain cleavage enzymes, dehydrogenase isomerases, β-reductases, α-reductases, oxidoreductases, hydroxylases, dehydrogenase/oxydoreductases, and epimerases. Additional examples of enzymes in the pathway include a P450 cholesterol side chain cleavage enzyme, hydroxysteroid dehydrogenase isomerases, β-reductases, hydroxysteroid oxidoreductases, hydroxylases, β-reductases, hydroxysteroid dehydrogenase/oxydoreductases, and hydroxysteroid epimerase. Particular enzymes in the pathway include P450 cholesterol side chain cleavage enzyme, Δ5-3-β-hydroxysteroid dehydrogenase isomerase, 5-β-reductase, 3-β-hydroxysteroid oxidoreductase, 5-β-hydroxylase, 5-α-reductase, 3-α-hydroxysteroid dehydrogenase/oxydoreductase, and 3-hydroxysteroid epimerase.

Other enzymes in the pathway include P450 cholesterol side chain cleavage enzyme, Δ5-3-β-hydroxysteroid dehydrogenase isomerase, 5-β-reductase, 3-β-hydroxysteroid oxidoreductase. Still other enzymes in the pathway include P450 cholesterol side chain cleavage enzyme and Δ5-3-β-hydroxysteroid dehydrogenase isomerase. One particular enzyme in the HIF pathway is subfamily XIA P450 cholesterol side chain cleavage enzyme. Another particular enzyme in the HIF pathway is Δ5-3-β-hydroxysteroid dehydrogenase isomerase.

A molecule in the biosynthetic pathway for use in the present invention can be obtained from a variety of sources. For example, the molecule can be obtained from a commercial source, purified (partially purified, substantially purified) or isolated from its natural environment, or synthesized.

A method of synthesizing a molecule (e.g., substrate or intermediate) in the HIF pathway includes isolating at least one enzyme from the pathway and employing the enzyme to perform a chemical transformation on the substrate. A particular embodiment includes employing at least two enzymes from the pathway, or more typically three enzymes. In other embodiments, when more than one enzyme is employed, the enzymes are selected to be adjacent in the sequence of the pathway. For example, a particular embodiment includes a P450 cholesterol side chain cleavage enzyme and a Δ5-3-β-hydroxysteroid dehydrogenase isomerase. In other embodiments, derivatives or related compounds of the substrates or intermediates are synthesized by employing the preceding combinations of enzymes to act on derivatives of the pathway substrates or intermediates, and/or employing different co-factors or consumable reagents.

The molecule in the biosynthetic pathway can be contacted with an agent to be assessed in a variety of ways known to those of skill in the art. For example, the molecule in the biosynthetic pathway can be contacted with the agent in vitro assay or in an in vivo assay.

A variety of methods can be used to determine whether the activity of a molecule in the biosynthetic pathway of HIF is altered when contacted with the agent. In one embodiment, whether the activity of the molecule in the biosynthetic pathway is altered is determined directly. For example, the function and/or expression of the molecule contacted with the agent is measured directly. In another embodiment, whether the activity of the molecule in the biosynthetic pathway is altered is determined indirectly. For example, the activity of a second molecule which is downstream of the molecule in the pathway (e.g., HIF) can be measured. Alteration of the activity of the second molecule is an indication that the activity of the molecule in the biosynthetic pathway is altered in the presence of the agent.

The present invention also relates to methods of identifying agents for treating conditions associated with HIF (e.g., abnormal or aberrant expression of HIF). In one embodiment, the present invention relates to a method of identifying an agent for treating hypertension. The method comprises contacting a molecule in a HIF biosynthetic pathway with an agent to be assessed, and determining the activity of the molecule in the presence of the agent. If the agent inhibits the activity of the molecule in the presence of the agent compared to the activity of the molecule in the absence of the agent, then the agent is identified as an agent for treating hypertension

As used herein, hypertension is an elevation of an organism's blood pressure compared to a control or a reference. Types of hypertension can include primary hypertension, i.e., essential or idiopathic hypertension; and secondary hypertension, e.g., hypertension attributable to stress, electrolyte balance (e.g., sodium, such as salt-sensitive hypertension), kidney (renal) disorder, renovascular hypertension, medication (e.g., decongestants, estrogen and derivatives, steroids asthma drugs, and the like), diet, smoking cholesterol levels, alcohol, age, physiological fitness, genetics, “white coat” hypertension, pregnancy-induced hypertension (e.g., including pre-eclampsia), and the like. Typically, hypertension includes essential hypertension, salt-sensitive hypertension, volume-expanded hypertension and secondary hypertension attributable to stress, electrolyte (e.g., sodium) intake, diet, smoking, cholesterol levels, physiological fitness, genetics, pre-eclampsia, and the like. More typically, hypertension includes essential hypertension, and secondary hypertension attributable to electrolyte (e.g., sodium) intake, diet, smoking, cholesterol levels, and physiological fitness. In a particular embodiment, hypertension is human essential hypertension.

In another embodiment, the present invention relates to a method of identifying an agent for treating heart failure (e.g., congestive heart failure), comprising contacting a molecule in the HIF biosynthetic pathway with an agent to be assessed and determining the activity of the molecule in the presence of the agent. If the agent enhances the activity of the molecule in the presence of the agent compared to the activity of the molecule in the absence of the agent, then the agent is identified as an agent for treating heart failure.

The methods of the present invention can further comprise use of a control or reference. A variety of controls are know to those of skill in the art. For example, a “control” includes a sample which is treated the same as the sample comprising the agent to be assessed, however, the sample does not include the agent to be assessed. In addition, the control sample can be a standard accepted as such in the industry or obtained from a commercial source. The control can be a model of the pathway; the control can be an in vitro system or an in vivo system. The control can be a previous state of the molecule or a desired state (future state; a state to be achieved) of the molecule.

The present invention also provides therapeutic methods for treating (ameliorating and/or preventing) conditions associated with HIF (e.g., associated with abnormal or aberrant activity of HIF). The therapeutic methods described herein can be used alone or in combination with other therapies used to treat conditions associated with HIF.

In one embodiment, the present invention relates to a method of treating hypertension in an individual, comprising administering to an individual in need thereof a therapeutically effective amount of an agent (one or more) that inhibits a molecule (one or more) in a biosynthetic pathway for hypothalamic inhibitory factor (HIF), thereby treating hypertension in the individual. In a particular embodiment, inhibition of the activity of the molecule in the pathway results in inhibition of the activity of HIF.

In another embodiment, the present invention relates to a method of treating heart failure in an individual, comprising administering to an individual in need thereof a therapeutically effective amount of an agent (one or more) that enhances a molecule (one or more) in a biosynthetic pathway for hypothalamic inhibitory factor (HIF), thereby treating heart failure in the individual. In a particular embodiment, enhancing the activity of the molecule in the pathway results in enhancement of the activity of HIF.

A “therapeutically effective amount” of one or more agents is administered to the individual by an appropriate route, either alone or in combination with another drug or treatment used to treat the condition associated with HIF activity (e.g., abnormal, aberrant activity of HIF) in an individual. A therapeutically effective amount is an amount sufficient to achieve the desired therapeutic effect when administered. For example, in some embodiments (e.g., method of treating hypertension), a therapeutically effective amount is an amount that is sufficient for inhibition of the activity of the molecule in the biosynthetic pathway of HIF, which results in inhibition of the activity of HIF. In other embodiments e.g., method of treating cardiac failure), a therapeutically effective amount includes an amount that is sufficient for promotion of the activity of the molecule in the biosynthetic pathway of HIF, which results in promotion of the activity of HIF.

The therapeutically effective amount will vary according to the condition being treated, the agent(s) being used, the formulation of the agent(s), the mode of administration and the age, weight and condition of the individual being treated. Dosages for a particular individual can be determined by one of ordinary skill in the art using conventional considerations (e.g., by means of an appropriate, conventional pharmacological protocol).

An agent of the invention is formulated (e.g., a pharmaceutical composition) to be compatible with its intended route of administration. Examples of routes of administration include parenteral, e.g., intravenous, intradermal, subcutaneous, oral, intranasal, transdermal (topical), transmucosal, and rectal administration (e.g., suppositories). The agents for use in the methods of the present invention can also include a sterile diluent such as water for injection, saline solution, fixed oils, polyethylene glycols, glycerine, propylene glycol or other synthetic solvents; antibacterial agents; antioxidants; chelating agents; buffers and agents for the adjustment of tonicity. The agent can be enclosed in ampoules, disposable syringes or multiple dose vials made of glass or plastic.

Pharmaceutical compositions of agents suitable for injectable use include sterile aqueous solutions (where water soluble) or dispersions and sterile powders for the extemporaneous preparation of sterile injectable solutions or dispersion. For intravenous administration, suitable carriers include physiological saline or phosphate buffered saline (PBS). The carrier can be a solvent or dispersion medium containing, for example, water, ethanol and polyol (e.g.,, glycerol, propylene glycol). In addition, a coating (e.g., lecithin) or a surfactant can be used. Antibacterial and antifungal agents, (e.g., thimerosal) can also be included. Moreover, sugars, polyalcohols and sodium chloride can be included in the pharmaceutical composition. An compound which delays absorption, for example, aluminum monostearate and gelatin can also be used.

Oral compositions can include an inert diluent or an edible carrier and can be in the form of capsules (e.g., gelatin), pills or tablets. The tablets, pills or capsules, can contain a binder, an excipient, a lubricant, a sweetening agent or a flavoring agent. For administration by inhalation, the agents are delivered in the form of an aerosol spray from pressured container or dispenser which contains a suitable propellant, e.g., a gas such as carbon dioxide, or a nebulizer.

In one embodiment, the active compounds can be administered as a controlled release formulation, including implants and microencapsulated delivery systems (e.g., biodegradable, biocompatible polymers can be used). Methods for preparation of such formulations will be apparent to those skilled in the art. The materials can also be obtained commercially.

The dosage of the pharamceutical compositions of the invention depend on the unique characteristics of the active compound and the particular therapeutic effect to be achieved, and the limitations inherent in the art of compounding such an active compound for the treatment of individuals.

Identification of a biosynthetic pathway of HIF also provides for diagnostic methods. In one embodiment, the present invention relates to a method of monitoring the effectiveness of a treatment of hypertension in an individual, comprising determining the activity of a molecule in a biosynthetic pathway for hypothalamic inhibitory factor (HIF) in an individual that has been treated, wherein if the activity of the molecule is inhibited and results in inhibition of HIF activity when the treatment is administered to the individual, compared to the activity of the molecule when the treatment is not administered to the individual, then the treatment is effective.

In another embodiment, the present invention relates to a method for assessing whether an individual is at risk for developing hypertension, comprising determining the activity of a molecule in a subject's biosynthetic pathway for hypothalamic inhibitory factor (HIF), wherein if the activity of the molecule is enhanced, thereby enhancing the activity of HIF in the individual, then the individual is at risk for developing hypertension.

The diagnostic methods of the present invention can be performed using a sample (e.g., a biological sample) from the individual. A “sample” includes biological samples such as tissues, cells, and biological fluids (e.g., urine, blood, lymph, spinal fluid) of a subject. The diagnostic methods can be performed in vitro, in vivo or in situ.

A diagnostic test for monitoring the treatment of hypertension in a subject includes determining the expression of (HIF) in the subject by determining the activity of a molecule in the subject's HIF biosynthetic pathway that is correlated to HIF expression. “Correlated to HIF expression” means that the level of the molecule and the expression of HIF are related, at least in part, by a linear or nonlinear correlation, typically related by a partial approximate linear correlation. The correlation can be either positive or negative. If the activity of the molecule indicates HIF expression is inhibited by treatment compared to HIF expression without treatment, the treatment is effective. If the activity of the molecule indicates HIF expression is not inhibited by treatment compared to the activity of the molecule without treatment, the treatment is ineffective. More typically, the correlation is positive, and if the activity of the molecule is inhibited by treatment compared to the activity of the molecule without treatment, the treatment is effective. If the activity of the molecule is not inhibited by treatment compared to the activity of the molecule without treatment, the treatment is ineffective.

A diagnostic test for assessing hypertension risk includes determining the expression of (HIF) in the subject by determining the activity of a molecule in the subject's HIF biosynthetic pathway that is correlated to HIF expression versus a control, e.g., a previous state of the subject, a population norm, a desired healthy state, and the like. In a particular embodiment, the control is a desired healthy state, i.e., a state generally recognized in the art as a desired state. In another particular embodiment, the control is a previous state in the subject, e.g., correlated to the subject's historical blood pressure, i.e., the risk factor can be determined as a function of time, and can include, for example, a simple comparison to a previous state, or cumulative/running predictive statistical models such as moving averages, linear projections, and the like. If the activity of the molecule versus time is enhanced, the subject's risk of hypertension is enhanced. If the activity of the molecule versus time is unchanged, the subject's risk of hypertension is unchanged. If the activity of the molecule versus time is inhibited, the subject's risk of hypertension is inhibited.

Typically, the agent alters the activity of the molecule by at least about 5% compared to the activity of the molecule in the absence of the agent, more typically by at least about 10%, still more typically by at least about 20%, at least about 30% and at least about 40%. In particular embodiments, the activity is altered by at least about 50% and more particularly about 75%. In a particular embodiment, the activity is substantially or totally inhibited, e.g, inhibited by at least about 90% or more particularly about 95%. In another particular embodiment, the activity is enhanced by at least about 10%, 20%, 30%, 40%, 50%, 75%, 90%, 95% and 100%; in yet another embodiment, the activity is enhanced by about 200%.

Described herein is characterization of a steroid-like endogenous, hypothalamus-derived inhibitor of Na+, K+-ATPase, i.e., OLC, and more particularly, HIF, including elucidation of its biosynthetic pathway.

These issues were addressed with a combination of molecular and genomic techniques. The in vivo model selected was the Milan hypersensitive rat strain, a genetically hypertensive animal which overproduces the inhibitor and whose pathophysiologic mechanisms parallel renal and cardiovascular events found in salt-sensitive human hypertension (Ferrandi et al., Acta Physiolog. Scandin., 168:187-193 (2000)). Furthermore, the Milan hypertensive strain (MHS) rat hypothalami contain 7-10 times more extractable HIF than the Milan normotensive strain (MNS) control rats, and a primary role for HIF is proposed in the pathogenesis of renal tubular Na+ transport abnormalities and the development of hypertension in this strain. Genomic analysis of these animal's production of the endogenous Na+ transport pump inhibitor was performed. This approach was selected to allow identification of key elements in the biosynthetic pathway of steroids, study of the occurrence of the relevant genes in brain and adrenal tissues, and study of expression abnormalities in the Milan hypertensive animal versus the Milan normotensive animal, which do not overproduce the Na+ pump inhibitor.

As shown in the following examples, cloning cDNAs following subtractive suppressive hybridization of MHS and MNS hypothalamic RNA yielded a gene coding for an enzyme fundamental in steroid biosynthetic pathway indicating that further application of this approach can lead to candidate genes which are expressed in the hypertensive brain tissue and which are likely related to production of HIF.

Exemplification

### EXAMPLE 1

### Ouabain-Like Compound in Milan Hypertensive Rats: Upregulation of Steroidogenic Genes in Hypothalamus but Not Adrenal

Bioinformatic Mapping of Crucial Genes in the OLC/HIF Biosynthetic Pathway; Identification of Enzymes in Rat Brain and Synthetic Intermediates

A bioinformatics approach was employed to map two novel biosynthetic pathways to OLC/HIF(FIG. 1). The goal was to identify enzymes in the pathway(s) which can add substituents to the steroid-cholesterol backbone, ultimately producing a candidate molecule for endogenous OLC/HIF. The general approach was to move from key words in relevant literature to a narrowing of search terms, detailed reading to refine terms, and search of several genome databases to identify known genes that can comprise elements of an enzymatic road map from cholesterol to ouabain or OLC/HIF. Several of these genes exist as members of a gene family, hence for some steps multiple candidate genes were identified. Data from many species, including plants, was employed to deduce these key enzymatic steps. The databases employed include, from the National Center for Biotechnology Information, Bethesda, Md.: LocusLink online at http://www.ncbi.nlm.nih.gov/LocusLink/index.html; GENBANK®, online at http://www.ncbi.nlm.nih.gov/Genbank/Genbank Overview.html; and dbEST, online at http://www.ncbi.nlm.nih.gov/dbEST/. Also used was GoldenPath (University of Santa Cruz, Santa Cruz, Calif.), available online at http://genome.ucsc.edu/.

Several of the relevant human genes for crucial steps in the pathway were identified, and these candidate genes, along with their identified rat orthologs are identified by the number key, common names, gene symbol, and Locus Link numbers (FIG. 1). However, several enzymes relevant to the pathway were not found in the databases, and were therefore cloned de novo.

Chemical intermediates that can be an important part of the pathway disclosed herein include the 5β-pregnane-3-β-ol-20-one and pregnane-3β, 5β-diol-20-one, because ouabain is hydroxylated in the β configuration at carbons 3 and 5. In fact, pregnane 3β, 5β-diol-20-one can be considered an important precursor in the pathway linking progesterone to the 5β-hydroxycardenolide known as strophanthidol. Strophanthidol is a molecule with close structural similarity to the aglycone of ouabain, ouabagenin, the latter differing only in two additional hydroxylations at carbons 1 and 11 (FIG. 1).

In addition to hydroxylation, additional biosynthetic steps can be involved in the complete synthesis of Oua, namely buteneolide formation (creation of the lactone ring at C17) and glycosylation at carbon 3. The database analysis provided candidate genes for some of these steps: 11β-hydroxylase (LocusLink 1585), which is almost identical in amino acid sequence to 11α-hydroxylase (the C11 hydroxyl is α in ouabain); cytochrome P450 19 (CYP XIX, 1588) which can serve to hydroxylate C19, or prepare it for hydroxylation; cytochrome P450 21 (CYP XXIA, 1589, 1590) which can catalyze a step in the formation of the lactone ring; and cytochrome P450 17 (CYP XVII, 1586). The latter has both 17α-hydroxylase and 17,20-lyase activities and can be a key enzyme in the steroidogenic pathway that produces progestins, mineralocorticoids, glucocorticoids, androgens, and estrogens.

Thus, this scheme can represent a gene-specific biochemical pathway for synthesis of OLC/HIF from cholesterol, and can provide the framework for expression analysis in hypertensive and normotensive Milan rat hypothalamus and adrenal.

Isolation of Rat RNA for Cloning cDNAs for Encoding Enzymes in the OLC/HIF Biosynthetic Pathway; Microarray Analysis of cRNA Targets in Rat Models

Previous data have shown that mature Milan hypertenisve rat (MHS) hypothalami have elevated levels of extractable HIF (˜10 fold) compared to normotensive (MHS), and that hypothalamic content of HIF from young (21 day old, prehypertensive) MHS exceeds that of mature MHS (5 month old, hypertensive). Adult MHS rats and their normotensive (MNS) controls were used. MHS and MNS rats were obtained from the internal stock colony (Prassis Sigma tau, Settimo Milanese, Italy). Rats were maintained under a controlled temperature of 22° C. and relative humidity of 55±10% with a 12 hour light/dark cycle. Rats were fed a regular standard diet (Altromin, Rieper, Vandois, Italy) and had free access to water. Systolic blood pressure (SBP) and heart rate were recorded weekly at the tail by plethysmography (BP recorder, U. Basile, Varese, Italy). SBP increases in MHS over MNS, starting from 4-5 weeks of age. At 3 months, SBP is significantly higher in MHS (168±0.9 mmHg) as compared to MNS (142±1.0 mmHg). Rats were sacrificed at 5 months by cervical dislocation. Immediately following sacrifice organs were removed (including hypothalamus, brain cortex, adrenal, kidney cortex, kidney medulla and liver), weighed and frozen in liquid nitrogen. RNA was extracted using TRIZOL® reagent according to the manufacturer's recommendations (Life Technologies, Gaithersburg, Md.) with representative gels showing clean bands of 18S and 28S RNA, characteristic of high quality RNA.

Using CODELINK® expression bioarray technology (Amersham Biosciences, Piscataway, N.J.), biotin-labeled cRNA targets were prepared from hypothalamic and adrenal total RNA from age matched male MHS and MNS rats, hybridized to the array and scanned following the manufacturer's specifications (Motorola Document 080045-00 Rev. 1, Northbrook, Ill.; now Amersham Biosciences, Piscataway N.J.). Scans were performed on a Perkin-Elmer HT 5000 using ScanArray Express software (Perkin-Elmer, Shelton, Conn.). Images were analyzed with CODELINK® Analysis version 2.1.17 (Amersham Biosciences). Comparison to hybridization intensities of negative control genes (bacterial) provided by the manufacturer established the threshold to conclude actual expression of studied genes in the respective tissues.

Microarray Analysis: Genes for P450 Cholesterol Side Chain Cleavage and Δ5-3β-HSD Isomerase are Differentially Expressed in Hypothalamus but not Adrenal.

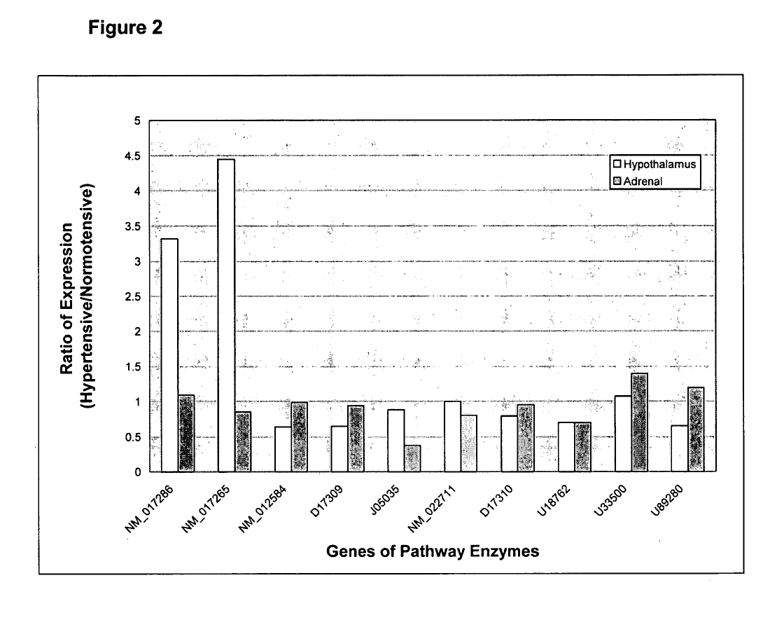

Working from the biosynthetic pathway(s) outlined in FIG. 1, rat orthologs to the human candidate genes were identified. Genes coding for seven of the enzymes were present on Amersham CODELINK® rat gene chips. All these genes were expressed in hypothalamic and adrenal tissues. Genes for five pathway enzymes showed no differential expression, while genes coding for the P450 cholesterol side chain cleavage (P450 scc, gene NM_{—}107286) and Δ5-3β-hydroxysteroid dehydrogenase isomerase (Δ5-3β-HSD, NM_{—}017265) enzymes showed 3.3 and 4.5-fold increased expression, respectively, in the hypertensive as compared to normotensive rat hypothalamus (FIG. 2). These enzymes are proximal in the biosynthetic pathway(s) with P450 scc catalyzing the conversion of cholesterol to pregnenolone, and Δ5-3β-HSD, pregnenolone to progesterone (FIG. 1).

Because both hypothalamus and adrenal have been considered as sources for OLC, adrenal RNA was analyzed from hypertensive and normotensive animals. The ratio of expression (MHS/MNS) of the P450 scc Δ5-3β-HSD was ˜1.0 by gene chip analysis, consistent with no differential expression (FIG. 2), although hybridization signals for both genes were very strong.

Ratio of expression of genes coding for P450 SCC
and Δ5-3β-HSD (MHS/MNS)

| | Hypothalamus | Adrenal |
|---|---|---|
| P450 SCC | 3.3 | 1.0 |
| Δ5-3β-HSD | 4.5 | 1.0 |

Quantitative, Real Time PCR Analysis

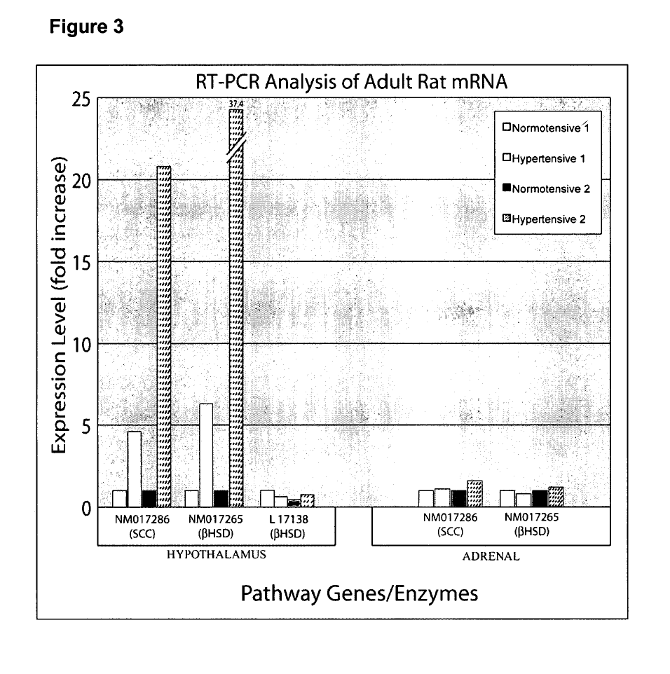

RT-PCR reactions were performed using an Real Time Quantitative PCR System 7700 (Applied Biosystems, Foster City, Calif.) according to the manufacturer's protocols. The primers used were designed with the software, PRIMER EXPRESS® provided by Applied Biosystems following the manufacturer's guidelines. Amplicons were about 100 bp in length and were amplified from primers with no significant secondary stem loop and homodimer structures. In addition, all primer sequences were screened with BLAST® analysis (NCBI) against rat genomic database to ensure that the primers designed were specific for the gene transcripts of interest. RT-PCR primer pairs used are (5′ to 3′): NM_{—}017286 (CAGGA CCTGG GCTCA ACTAT G (SEQ ID NO: 1) and AGAGA CACCA CCCTC AAATG C (SEQ ID NO: 2)), NM_{—}017265 (CCAGC TAGGA CAGAG GCACA AT (SEQ ID NO: 3) and ATTAG GGAAG AAAGC TTGTG GACTA G (SEQ ID NO: 4. For each experimental sample 2 μg of total RNA was reverse transcribed with Multiscribe (Applied Biosystems) and random hexamers as primer. After quantification, 10 ng cDNA from each tissue sample was used for amplification with primer pairs for each of the genes of interest plus one reaction with primers for the housekeeping gene GAPDH as internal control. The RT-PCR reactions were carried out under the universal cycling conditions (95° C. 10 min, 68° C. 2 min, 72° C. 10 min, 34 cycles) and the data were processed with the software Sequence Detector (Applied Biosystems). Following normalization to the control, the abundance of each gene transcript in different tissue samples was expressed as fold difference over the abundance of the same gene transcript in the paired normotensive rat. Results are shown in FIG. 3.

Quantitative (Real Time)-PCR Analysis Confirms Differential Expression of P450 scc and Δ5-3β-HSD in Hypertensive Hypothalamus Parallel to Microarray Analysis.

FIG. 3 shows data from hypothalamic RNA of two different hypertensive and normotensive animal pairs, corresponding to the animals for which the microarray data were obtained. In the first compared pair (animals 1), genes coding for P450 scc and Δ5-3β-HSD were up-regulated 4 and 6-fold, respectively, in the hypertensive hypothalamus. For the second pair of animals (animals 2), the up-regulation was even more marked, showing 21 and 37.4-fold increases for the respective genes in the hypertensive brain.

Although both genes are expressed in adrenal tissue as expected, quantitative real time-PCR analysis showed no significant differential expression for genes coding for P450 scc and Δ5-3β-HSD in hypertensive rat adrenal tissue (FIG. 3), confirming the microarray data (FIG. 2).

Discussion

Using a bioinformatics approach, steroid biosynthetic pathway(s) leading from cholesterol as a precursor through intermediate hydroxylation steps consistent with generation of a steroid intermediate(s), which after lactone ring addition and glycosylation results in generation of Oua or its isomer, were generated (FIG. 1). Rat orthologs of seven potentially relevant human genes in the early steps for this pathway were identified and found to be available for microarray analysis on the CODELINK® rat gene chips. All ortholog genes were expressed in both hypothalamus and adrenal tissues relative to negative control threshold. Chip analysis of paired MHS and MNS RNA isolates showed no differential expression of genes encoding five of the enzymes, but revealed significant overexpression in hypertensive hypothalamus for genes coding for P450 side chain cleavage and Δ5-3β-hydroxysteroid dehydrogenase isomerase, the first two enzymes in the hypothetical pathway (FIGS. 1, 2). Interestingly, P450 scc is felt to be generally rate-limiting in the biosynthesis of steroid hormones in established classical synthetic pathways (Orth, D. N., et al., Williams Textbook of Endricrinology, Philadelphia, W B Saunders, 1998, pp. 517-564).

It is likely that all enzymes needed to synthesize HIF/OLC are present in adrenal and hypothalamic tissues and that all relevant genes are expressed as expected since both tissues produce HIF/OLC. However, unlike hypertensive hypothalamic tissue, gene chip analysis of RNA isolates from the corresponding animal adrenal tissues showed no difference in expression between hypertensive and normotensive animals (FIG. 2).

Quantitative (real time) PCR analysis of the same RNA isolates from hypertensive and normotensive tissues was used to confirm the microarray data. Genes NM_{—}017286 (coding for P450 scc) and NM_{—}017265 (Δ5-3β-HSD) in MHS hypothalamus showed marked overexpression compared to normotensive (MNS) hypothalamus, confirming results of the microarray analysis (FIG. 3). Thus, differential expression of genes governing the conversion of cholesterol to pregnenolone and pregnenolone to progesterone are confirmed by RT-PCR in hypertensive hypothalamus.

Although expressed, quantitative PCR analysis showed no significant differential expression for these genes in hypertensive rat adrenal tissue (FIG. 3).

Structual studies following tissue extraction and purification have established forebrain (hypothalamus) and adrenal as the favored tissue sources for OLC. In the case of rats, the endogenous origin of OLC isolates was questioned in the past since in certain instances, rat chow could be identified as a source of OLC (Tamura, M., et al., J. Biol. Chem., 269:11972-11979 (1994)), and rat adrenal tissue could be shown to accumulate tritium-labeled Oua when added to the diet (Kitano et al., Hypertens. Res., 21:47-56 (1998)). Importantly for our studies, tissue contents of OLC in Milan rats were shown to be unaffected by rat chows containing varying amounts of extractable Na⁺ transport inhibitors (Ferrandi, M., et al., J. Hypertens., 13:1571-1574 (1995)).

A limited number of biosynthetic studies have addressed the issue of endogenous production. Radioimmunoassay analyses using polyclonal anti-ouabain antibodies indicated that OLC can be secreted by adrenal cortical cells in culture in response to receptor stimulation (Laredo, J., et al., Hyperten., 29:401-407 (1997)) and feeding of steroid hormone precursors (Perrin, A., et al., Mol. Cell Endrocrinol., 126:7-15 (1997)). Lichstein and co-workers demonstrated that rat adrenal homogenates fed radiolabeled hydroxycholesterol incorporate the radiolabel in a compound(s) with chromatographic retention time and bioactivity characteristic of OLC, and that side chain cleavage is the first step in the synthesis of these digitalis-like compounds (Lichtstein, D., et al., Life Sci., 62:2109-2126 (1998)).

Issues of antibody non-specificity in the identification of OLC in earlier studies were circumvented by Perrin, A., et al., Mol. Cell Endrocrinol., 126:7-15 (1997) (adrenal cortex) and Komiyama, Y., et al., J. Hypertens., 19:229-236 (2001) (adrenal medulla) who carried out physico-chemical analysis of immunoreactive isolates from the cell culture supernatants. Liquid chromatography-mass spectrometry analysis in both instances showed the released OLC to have a molecular mass identical to plant Oua (Perrin, A., et al., Mol. Cell Endrocrinol., 126:7-15 (1997); Komiyama, Y., et al., J. Hypertens., 19:229-236 (2001)). Since NMR analysis was not done, the authors could not say whether the stereochemistry of the respective isolates was that of Oua or its isomer.

While similar studies of hypothalamus/midbrain are not available, it has been demonstrated that enzymatic pathways for de novo biosynthesis of “neurosteroids” exist in brain, including key enzymes to generate pregnenolone, progesterone and distal metabolites which could serve as precursors in a pathway leading to cardiac glycoside-type compounds (Robel, P., et al., Crit. Rev. Neurobiol., 9:383-394 (1995)). We previously found in normal Wistar rats that exposure to low oxygen tension in vivo and in vitro markedly enhanced the release of a Na⁺ transport inhibitor co-chromatographing with HIF, from hypothalamus slices incubated in vitro, and this release was further increased by exposure of the brain tissue to high NaCl concentrations (De Angelis, C., e al., Am. J. Physiol., 274:F182-F188 (1998)). Conversely, atrial natriuretic peptide injected intravenously or included in the in vitro incubation of rat hypothalamic tissue decreased the release of the Na⁺, K⁺-ATPase inhibitor (Crabos, M., et al., Am. J. Physiol., 254:F912-F917 (1988)). The time course of some of these experiments would have allowed for the possibility of de novo synthesis vs. release of stores in response to the physiologic and pharmacologic manipulations, but this issue was not specifically addressed in the studies.

There are no direct studies of specific gene expression related to the biosynthesis OLC in putative source tissues. The findings reported here of overexpression of P450 scc- and β-HSD-associated genes is compatible with delivery of enhanced substrate intermediates to the corticosterone biosynthetic branch in MHS hypothalamus. This supports the observation that treatment of adrenal tissues with cyanoketone, which blocks Δ5-3β-HSD activity, was accompanied by decreased production of immunoreactive OLC by the cells as measured by radioimmunoassay (Lu Z-R., et al., Hypertension, 32(3):624 (1998)). As shown below, the functional relevance of the overexpression of these genes was confirmed by knockdown of one of the genes, which was accompanied by decreased mRNA levels and diminished release of HIF/OLC into culture supernatants of cells documented to release OLC. In this regard, preliminary studies confirm the report of Komiyama et al. (Komiyama, Y., et al., J. Hypertens., 19:229-236 (2001)) that PC12 adrenal medullary cells (tissue of neural origin) release Na⁺ transport inhibitory activity into supernatants, and that genes NM_{—}017286 (P450 scc) and NM_{—}017265 (Δ5-3β-HSD) are expressed in the cells as measured by RT-PCR.

It was not surprising that microarray analysis of whole adrenal RNA did not reveal differential expression of P450 scc and Δ5-3β-HSD genes. Both genes in adrenal are so highly expressed that a significant increase, emanating from either a cortical or medullary portion, could be masked. The expression analysis reported here therefore does not exclude the possibility that adrenal cortex is involved in OLC biosynthesis, though it indicates that adrenal enzyme levels are not altered in Milan hypertensive rats.

MHS hypothalamic tissue shows a dramatically different pattern, with marked over expression in hypertensive hypothalamus of two key genes in steroid biosynthesis. These findings indicate that a unique steroid biosynthetic circuit exists in MHS hypothalamus, functioning independently from classic adrenal cortical pathways, to provide substrate for a branch leading to HIF production which would account for the increased extractable levels of this endogenous Na⁺, K⁺-ATPase inhibitor now linked to the pathogenesis of hypertensive disease in this strain.

There is no information available about biosynthesis of the hypothalamic ouabain-like compound (“HIF”; hypothalamic inhibitory factor), or of a gene-specific biosynthetic pathway for the substance in either hypothalamus or adrenal. Milan hypertensive rats were chosen for analysis since this strain has elevated levels of HIF in hypothalamus, and this Na⁺ pump inhibitor is directly implicated in the pathogenesis of hypertension. We approached the issue by constructing a pathway from sequence data bases, and used microarrays to investigate expression of specific genes, with hits confirmed by quantitative PCR. Genes for cholesterol side chain cleavage (P450 scc) and hydroxysteroid dehydrogenase isomerase enzymes were upreguated in hypertensive hypothalamus but not adrenal, indicating an independent steroid biosynthetic circuit in hypertensive brain. Since P450 scc is the rate-limiting step in classical steroid biosynthesis, the genetic upregulation in hypertensive hypothalamus likely results in enhanced substrate for downstream conversions leading to increased HIF production. Identification of genes important in HIF production provide insights into pathogenesis and novel therapeutic targets for the treatment of hypertension.

### EXAMPLE 2

### RNA Interference Studies

siRNAs were designed using several well-described principles (see Fire et al., Nature 391(6669):806-811 (1998); McManus and Sharp, Nature Review Genetics, 3(10):737-747 (2002)). The siRNA duplexes are 21 nucleotides in length, with a 2-nucleotide 3′ overhang and a GC content of 50% or less. The target sequences are located at least 50 nucleotides following the start codon. All sequences were checked by BLAST to ensure that our gene of interest was exclusively targeted. These siRNAs were synthesized by Xeragon (now Qiagen).

PC12 cells were passaged 24 hours before transfection for an optimal 80% confluency on the day of transfection. They were grown in DMEM containing 10% horse serum, 5% fetal calf serum, 100 U/ml penicillin, and 100 μg/μl streptomycin (referred to as growth medium).

PC12 cells were transfected with siRNA duplex using TransMessenger Reagent (Qiagen). For one well of a 6-well tray, 0.84 μg of siRNA duplex (mixing 2 μl of enhancer R with 94 μl of buffer EC and 4 μl of siRNA duplex) was used, the mix was vortexed, and incubated 5 minutes at room temperature. Then the appropriate amount (differing ratios) of TransMessenger was added to the same tube, vortexed, and incubated 10 minutes at room temperature. After washing the PC12 cells with PBS (containing CaCl₂ and MgCl₂), 900 μl of DMEM (without serum or antibiotics) was added to the tube, mixed and transferred to the well of PC12 cells. After 2-4 hours, 1 ml of growth medium was added to the cells without removing the transfection medium. After 1-2 days conditioned medium and RNA was collected from cells.

After purifying the conditioned medium by reverse-phase column chromatography (Sep-Pak, Waters), the resulting sample was assayed using the ⁸⁶Rb uptake assay to determine a change in inhibition as previously described in Carilli et al., J. Biol. Chem., 260:1027-1031 (1985).

Further, after purifying the RNA (RNeasy, Qiagen), real time PCR (RT-PCR) was performed as reviewed in Klein, Trends in Molec. Med.,8(6):257-260 (2002). Briefly, the RNA was reverse transcribed and this was used as the template for PCR. The template was mixed with the polymerase, buffer, primers and sybr green dye. Increase in fluorescence was monitored over the course of 40 rounds of amplification on the ABI PRISM 7700 (Applied Biosystems). A no template control was used to assess background levels of amplification. Amplification by target primers and control primers were evaluated to determine a fold change.

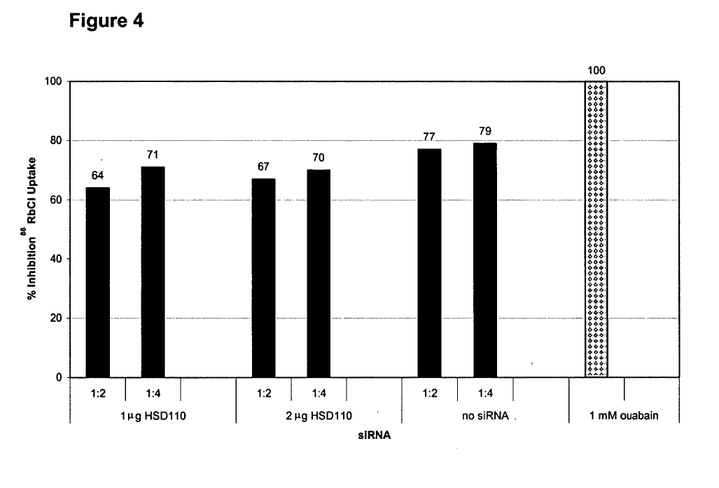

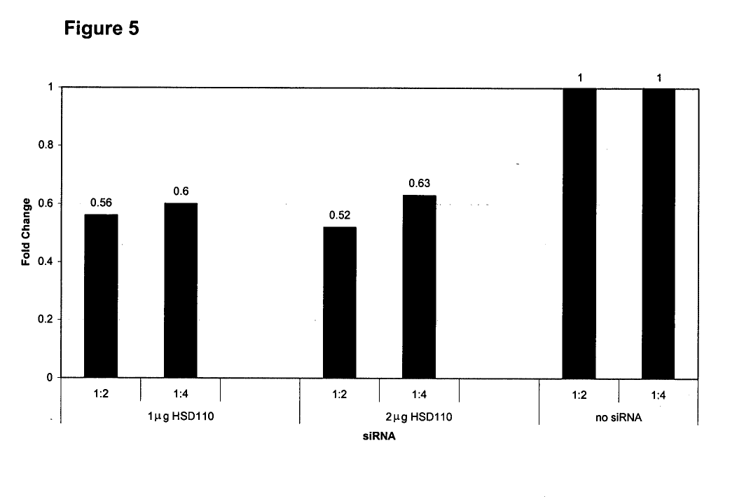

Results are shown in FIGS. 4 and 5. FIG. 4 shows knock down of HIF activity purified from cultured PC12 (adrenal medulla) cells transfected with siRNA (HSD110) targeting gene Δ-5-3β-hydroxysteroid dehydrogenase isomerase (NM_{—}017265). HIF was purified from PC12 cell supernatants by column chromatography. Bioactivity is demonstrated by inhibition of Na⁺ pump activity (active Rb⁺ uptake) in human erythrocytes (Carilli et al, 1985). Cells treated with siRNA HSD110 synthesized less HIF as indicated by decrease in inhibitory activity. X-axis reflects optimization studies varying the amount of siRNA (μg) and ratio of siRNA to transfection reagent. Complete inhibition of active Na⁺ transport is caused by 1 mM ouabain as a standard.

FIG. 5 shows the decrease in Δ-5-3β-hydroxysteroid dehydrogenase isomerase mRNA levels in PC12 (adrenal medulla) cells transfected with siRNA HSD110 as measured by quantitative reverse transcriptase polymerase chain reaction. X-axis reflects optimization studies varying the amount of siRNA (μg) and ratio of siRNA to transfection reagent. Decrease in mRNA was accompanied by decreased production of HIF.

### EXAMPLE 3

### Identification of Additional Biosynthetic HIF Enzymes

Suppressive, Subtractive Hybridization and Cloning of cDNA from MHS Hypothalamic RNA to Identify Candidate HIF Biosynthetic Enzymes

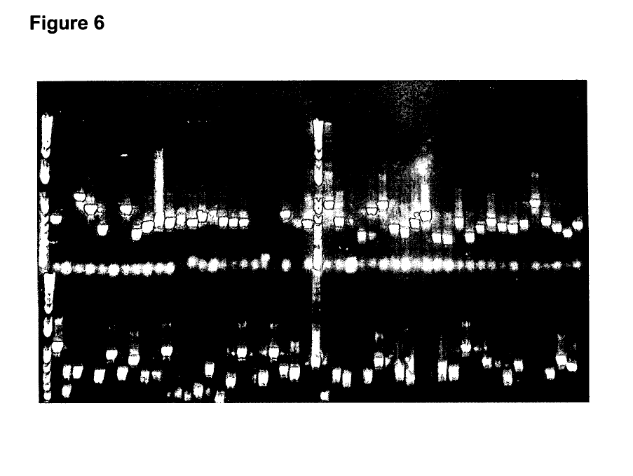

It is likely that MHS animals overexpress genes encoding enzymes in the HIF pathway as compared to MNS animals. In addition to using RT-PCR and microarrays to identify key genes, a subtractive hybridization strategy is also used. As a method to enrich differentially expressed low abundance transcripts, suppressive subtractive hybridization (“subtractive cloning”) was used. In this technique, cDNAs from MHS and MNS hypothalamic total RNA were first made and amplified (reverse transcriptase/polymerase chain reaction) using the Clontech Smart PCR cDNA Synthesis Kit. The concept behind the subtractive cloning technique is that DNA common to both the hypertensive and normotensive hypothalami is eliminated (“subtracted out”), leaving one with differentially expressed genes associated with the hypertenisve rat brain tissue. Thus, MHS and MNS cDNAs from the Clontech Smart method were subjected to subtraction using the Clontech PCR-Select cDNA Subtraction kit. The subtracted DNA was then cloned using TOPO TA (Invitrogen) and UA (Qiagen) cloning methodologies. Random colonies of transformed bacteria were selected for plasmid isolation, gel electrophoresis for identification of cloned inserts, and sequence analysis. The cloning procedure was carried out in a 96 well format. FIG. 6 shows agarose gel electrophoresis of DNA inserts from the subtracted MHS cDNA (second cloning). Lanes 1, 26 and 51 show lkb DNA ladders for reference. DNA corresponding to selected (differing) base pair numbers was subcloned, DNA isolated, and purified for sequencing.

Sequence information for one cDNA clone has been obtained and 76 additional clones are being analyzed. BLAST analysis showed that this cDNA is most homologous to a key enzyme in the steroid biosynthetic pathway, lanosterol synthetase (2,3-oxidosqualene-lanosterol cyclase). This enzyme cyclizes squalene to form lanosterol which is a principal precursor of cholesterol, the latter lying at the head of the pathway for HIF described herein (FIG. 1).

Clone and Sequence of Rat Orthologs of Human cDNAs to Recapitulate the Putative Steroid Biosynthetic Path in Rat Brain

Starting with key intermediates outlined in FIG. 1, primers to the known candidate genes essential to the pathway are selected and designed. It is likely that there are about 10-12 enzymes in the biosynthetic process of which eight candidates have been identified. In cases where the rat cDNA is known, the appropriate transcript is sequenced using primers that encompass the entire mRNA and/or coding region. In the instances where the rat sequence for a candidate cDNA is not available, degenerate primers based on human and/or mouse sequences are used to clone at least a partial length cDNA. Full-length cDNAs are obtained using RACE or library screening. Following reverse transcription of total RNA from MHS and MNS hypothalamus and adrenal (initially), aliquots of cDNA are divided and respective primers added for PCR. Candidates for subcloning are selected following agarose gel electrophoresis and sequence verification. Also a combination of low stringency cloning and bioinformatics is used.

Characterize Baseline and Differential Expression of Candidate Genes in Target Tissues

Candidate genes are characterized using Northern blot and quantitative RT-PCR in rat tissue from hypertensive and normotensive animals. It is likely that DNA will not be expressed in all tissues, and expression levels will be enriched in organs that make OLC/HIF, specifically, brain and adrenal.

Additionally, expression of the candidate genes in PC12 (rat adrenomedullary) cells which are known to synthesize OLC/HIF is demonstrated and used as a functional assay to validate a role for candidate genes in HIF biosynthesis.

Identify New Candidate Genes Missing in the Biosynthetic Pathway

cDNAs from MHS tissue RNA is cloned following subtractive, suppressive hybridization. Individual cDNAs are isolated and sequenced. Microarray analysis (gene chip technology) is also employed. A DNA microarray is a matrix of thousands of cDNA or oligonucleotides imprinted on a solid support. Labeled cRNA from a tissue of interest is hybridized to its sequence complement on the array to provide a measure of the mRNA abundance in the sample. The pattern of gene expression is analyzed by statistical and bioinformatic analysis. Commercial high density microarrays containing thousands of genes on one chip are available for rat genes, including chips containing genes derived from brain tissue (e.g., Affymetrix A/B/C/chips; Motorola arrays). Such microarrays are used to hybridize labeled MHS and MNS cRNA. The data is used to identify genes based on tissue distribution and differential expression profiles in MHS versus MNS animals.

Subtraction cloning with customized gene chips is also used. Customized chips represent arrays of cDNA or oligonucleotides chosen from targeted biological sources (or public or institutional repositories), and spotted robotically in a defined matrix, e.g., on a glass slide. DNA from a cloned “hypertensive” library are used to make customized chips to probe RNA from hypertenisve and normotensive animals. In addition, oliogs or cDNA that represent differentially expressed transcripts identified from analysis of commercial arrays so that they can be arrayed on the custom chips are obtained. The candidate DNA is expressed in steroid producing tissues (e.g., hypothalamus and adrenal), but not in all tissues (e.g., skeletal muscle), and is differentially expressed in MHS.

Microarrays provide “static” data, that is, comparisons of two independent samples (e.g., MHS versus MNS); and “dynamic” data, in which temporal (time line) gene expression changes are monitored from a single sample. This is useful because young, prehypertenisve MHS have higher hypothalamic HIF content than mature MHS with established hypertension.

Functional Verification of Candidate Genes: Knock-Out Studies

PC12 cells (adrenal medullary cells) release an OLC into culture supernatants, and the amount of release can be augmented in a dose-dependent manner by adding progesterone to the culture media (Komiyama et al., J. Hypertens., 19:229-236 (2001)). The release of HIF from adrenal incubated as tissue slices (DeAngelis, et al., Am. J. Physiol., 274:F182-188 (1998)) which did not separate cortical from medullary portions has been previously demonstrated. PC12 cells are used to study the effects of genetic manipulation on OLC/HIF production. PC12 cells are studied for the presence of the genes of interest, then the release of inhibitor from these cells is confirmed using the method of Komiyama (Komiyama et al., J. Hypertens., 19:229-236 (2001)). Collected supernatants are acidified to dissociate OLC from putative carrier proteins, and purified using Sep-Pak C18 cartridges eluted with acetonitrile. The purified eluate is assayed for Na⁺ pump inhibitory activity by measuring active ⁸⁶Rb⁺(K⁺ analogue) transport into human erythrocytes, a standard method for detecting and quantifying HIF activity (DeAngelis, et al., Am. J. Physiol., 274:F182-188 (1998)). The PC12 released inhibitor is also tested for chromatographic retention time in comparison with HIF using lipophilic gel and high performance liquid chromatography since retention times for HIF is established in these chromatographic systems (Tymiak et al., PNAS, 90:8189 (1993)).

Northern blot and/or RT-PCR analysis is used to confirm that the genes are expressed in PC12 cells. The genes are knocked out individually using RNA interference (RNAi). In this technique, double stranded RNA matching a target gene sequence is synthesized in vitro, cleaved with ribonuclease to produce short interfering RNA duplexes, these latter introduced into cells targeting the mRNA of the gene to be silenced.

Using RNAi methods, each gene is individually “knocked out” in PC12 cells. Time course studies are used to analyze mRNA levels, which are known to be suppressed using RNAi, to identify the optimal time of suppression. Cell supernatants are assayed for HIF activity. In addition, studies to identify conditions that enhance or suppress synthesis of HIF, including hypoxia which stimulates (DeAngelis, et al., Am. J. Physiol., 274:F182-188 (1998)) and atrial natriuretic peptide which suppresses (Crabos, et al., Am. J. Physiol., 254 (Renal Fluid Electrolyte Physiol., 23):F912-F917 (1988)) HIF, are performed, and whether there is coordinate regulation of the key enzymes is determined.

The purified protein of a cDNA identified is expressed and its biochemical activity is demonstrated.

Each reference cited herein is incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying an agent that alters the activity of hypothalamic inhibitory factor (HIF), comprising:
a) contacting a molecule in a biosynthetic pathway for HIF with an agent to be assessed; and
b) determining the activity of the molecule in the presence of the agent;
wherein, if the agent alters the activity of the molecule in the presence of the agent compared to the activity of the molecule in the absence of the agent, then the agent alters the activity of HIF.

2. The method of claim 1, wherein the agent inhibits the activity of the molecule.

3. The method of claim 1, wherein the agent enhances the activity of the molecule.

4. The method of claim 1, wherein the agent is selected from the group consisting of: a peptide, a steroid, and an antibody.

5. The method of claim 1, wherein the molecule is selected from the group consisting of: P450 cholesterol side chain cleavage enzyme and Δ5-3-β-hydroxysteroid dehydrogenase isomerase.

6. A method of identifying an agent for treating hypertension, comprising:
a) contacting a molecule in a biosynthetic pathway for hypothalamic inhibitory factor (HIF) with an agent to be assessed; and
b) determining the activity of the molecule in the presence of the agent;
wherein, if the agent inhibits the activity of the molecule in the presence of the agent compared to the activity of the molecule in the absence of the agent, then the agent is identified as an agent for treating hypertension.

7. The method of claim 6 wherein the agent can be used to treat hypertension selected from the group consisting of: secondary hypertension, salt-sensitive hypertension, volume-expanded hypertension, pregnancy-induced hypertension and essential hypertension.

8. The method of claim 6, wherein the agent is selected from: a peptide, a steroid, and an antibody.

9. The method of claim 6, wherein the molecule is selected from P450 cholesterol side chain cleavage enzyme and Δ5-3-β-hydroxysteroid dehydrogenase isomerase.

10. A method of identifying an agent for treating heart failure, comprising:
a) contacting a molecule in a biosynthetic pathway for hypothalamic inhibitory factor (HIF) with an agent to be assessed; and
b) determining the activity of the molecule in the presence of the agent;
wherein, if the agent enhances the activity of the molecule in the presence of the agent compared to the activity of the molecule in the absence of the agent, then the agent is identified as an agent for treating hypertension.

11. The method of claim 10, wherein the agent is selected from the group consisting of: a peptide, a steroid, and an antibody.

12. The method of claim 10, wherein the molecule is selected from P450 cholesterol side chain cleavage enzyme and Δ5-3-β-hydroxysteroid dehydrogenase isomerase.

13. A method of treating hypertension in an individual, comprising administering to an individual in need thereof a therapeutically effective amount of an agent that inhibits a molecule in a biosynthetic pathway for hypothalamic inhibitory factor (HIF), thereby treating hypertension in the individual.

14. The method of claim 13 wherein the hypertension is selected from the group consisting of: secondary hypertension, salt-sensitive hypertension, volume-expanded hypertension, pregnancy-induced hypertension and human essential hypertension.

15. The method of claim 13, wherein the agent is selected from: a peptide, a steroid, and an antibody.

16. The method of claim 13, wherein the molecule is selected from the group consisting of: P450 cholesterol side chain cleavage enzyme and Δ5-3-β-hydroxysteroid dehydrogenase isomerase.

17. A method of treating heart failure in an individual, comprising administering to an individual in need thereof a therapeutically effective amount of an agent that enhances a molecule in a biosynthetic pathway for hypothalamic inhibitory factor (HIF), thereby treating heart failure in the individual.

18. The method of claim 17, wherein the agent is selected from: a peptide, a steroid, and an antibody.

19. The method of claim 17, wherein the molecule is selected from P450 cholesterol side chain cleavage enzyme and Δ5-3-β-hydroxysteroid dehydrogenase isomerase.

20. A method of monitoring the effectiveness of a treatment of hypertension in an individual, comprising determining the activity of a molecule in a biosynthetic pathway for hypothalamic inhibitory factor (HIF) in an individual that has been treated, wherein if the activity of the molecule is altered and results in inhibition of HIF activity when the treatment is administered to the individual, compared to the activity of the molecule when the treatment is not administered to the individual, then the treatment is effective.

21. The method of claim 20, wherein the molecule is selected from P450 cholesterol side chain cleavage enzyme and Δ5-3-β-hydroxysteroid dehydrogenase isomerase.

22. A method for assessing whether an individual is at risk for developing hypertension, comprising determining the activity of a molecule in a subject's biosynthetic pathway for hypothalamic inhibitory factor (HIF), wherein if the activity of the molecule is altered, thereby enhancing the activity of HIF in the individual, then the individual is at risk for developing hypertension.

23. The method of claim 22, wherein the molecule is selected from P450 cholesterol side chain cleavage enzyme and Δ5-3-β-hydroxysteroid dehydrogenase isomerase.


SEQUENCE LISTING (4 sequences)

SEQ ID NO: 1 - DNA, 21 bp, Artificial Sequence
  NM-017286 primer
caggacctgggctcaactatg

SEQ ID NO: 2 - DNA, 21 bp, Artificial Sequence
  NM-017286 primer
agagacaccaccctcaaatgc

SEQ ID NO: 3 - DNA, 22 bp, Artificial Sequence
  NM-017265 primer
ccagctaggacagaggcacaat

SEQ ID NO: 4 - DNA, 26 bp, Artificial Sequence
  NM-017265 primer
attagggaagaaagcttgtggactag